(19) United States
(12) Patent Application Publication
(10) Pub. No.: US 2005/0059719 A1
(43) Pub. Date: Mar. 17, 2005

(54) Solid dosage formulation containing a Factor Xa inhibitor and method

(76) Inventors: Sherif Ibrahim Farag Badawy, Dayton, NJ (US); Munir Alwan Hussain, Belle Mead, NJ (US); Duxin Sun, Dublin, OH (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/924,043

(22) Filed: Aug. 23, 2004

Related U.S. Application Data
(60) Provisional application No. 60/503,330, filed on Sep. 16, 2003.

(51) Int. Cl. A61K 31/42

(52) U.S. Cl. 514/379

(57) ABSTRACT

An oral solid dosage formulation is provided which contains a Factor Xa inhibitor for which oral bioavailability is not reduced by co-administration of antacids, H2 antagonists and proton pump inhibitors. Such solid dosage formulation includes the Factor Xa inhibitor of the structure, 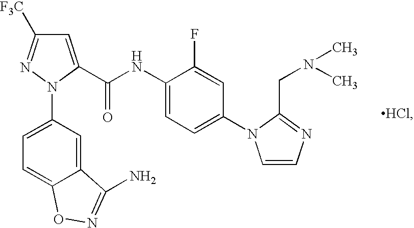 a pharmaceutically acceptable carrier, and an acid component, such as tartaric acid, whereby upon ingestion of the oral solid dosage formulation, the acid component increases solubility of the Factor Xa inhibitor in the local environment of the dissolving solid dosage formulation resulting in an otherwise lower degree of supersaturation of the Factor Xa inhibitor in such environment, than if the acid were not present. The result is that precipitation of the Factor Xa inhibitor in the form of its insoluble free base is minimized during dissolution of the Factor Xa inhibitor thereby increasing its oral bioavailability. A method for enhancing bioavailability of the Factor Xa inhibitor is also provided wherein an acid such as tartaric acid is incorporated with the solid dosage pharmaceutical carrier for the Factor Xa inhibitor.

### FIELD OF THE INVENTION

This application claims a benefit of priority from U.S. Provisional Application No. 60/503,330, the entire disclosure of which is herein incorporated by reference.

The present invention relates to an oral solid dosage formulation, preferably a tablet, containing a medicament which is a Factor Xa inhibitor, which formulation provides for enhanced oral bioavailability for the medicament, even when administered with antacids and/or H2 antagonists, due to the presence of an acid in the formulation; to a method of enhancing bioavailability of an oral solid dosage form containing the medicament, when the dosage form is administered with antacids and H2 antagonists, by including an acid component therein; and to a method for preparing the formulation.

### BACKGROUND OF THE INVENTION

U.S. Pat. No. 6,339,099 discloses the Factor Xa inhibitor 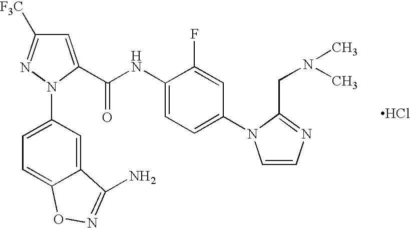
(hereinafter referred to as the Factor Xa inhibitor) which is a weak base with pH dependent solubility which shows decrease in solubility as the pH is increased. The neutral form or free base of the Factor Xa inhibitor has extremely low solubility, which is estimated to be 0.1 μg/mL. Moreover, the Factor Xa inhibitor in the form of its hydrochloride salt, at normal gastric pH condition, where the pH of the gastric medium is ˜1-2, has a solubility of ˜3 mg/mL. As a result, an entire dose of the Factor Xa inhibitor (up to 100 mg) is expected to dissolve completely in the gastric medium. Consequently, the Factor Xa inhibitor is well absorbed in fasted humans, and plasma AUC is proportional to the dose when administered as a Drug in Bottle (solution/suspension) formulation. Also, traditional immediate release tablets containing the hydrochloride salt showed identical pharmacokinetic profile to the Drug in Bottle formulation.

However, significant reduction in plasma AUC was observed when the immediate release tablets containing the Factor Xa inhibitor in the form of it's hydrochloride salt were co-administered with H2 receptor antagonists or antacids in human subjects and in dogs. The co-administration of these agents results in an increase in gastric pH up to 5 or more. In-vitro studies indicate that the hydrochloride salt undergoes conversion to the free base during dissolution in aqueous media at pH 5.5. As the solubility of the hydrochloride salt is limited by the solubility of the free base at this pH, a highly supersaturated solution with respect to the free base forms as the salt is dissolving, from which the free base precipitates out. Since the free base has very low solubility, absorption of the Factor Xa inhibitor becomes limited by the slow dissolution rate of the free base resulting in the AUC reduction observed when co-administered with H2 receptor antagonists or antacids.

In-vitro dissolution testing of a conventional tablet formulation containing the Factor Xa inhibitor in the form of it's hydrochloride salt, 100 mg strength, in acetate buffer at pH 5.5 showed rapid dissolution rate initially where approximately 35-40% of the dose dissolved in 10 minutes. However, there was no further increase in the fraction dissolved beyond the 10 minute time point and the percent dissolved remained constant after 3 hours. Factor Xa inhibitor concentration in the dissolution medium was ˜10 fold higher than its saturation solubility at this pH indicating that drug can exist in a supersaturated solution for a relatively extended time period under these conditions. The lack of complete dissolution is believed to be due to the conversion of the undissolved fraction to the free base as the hydrochloride salt is initially dissolving. Thus, while the Factor Xa inhibitor showed the ability to maintain a supersaturated solution in the bulk dissolution medium for a relatively long time period, the significantly higher degree of supersaturation in the local environment of the dissolving tablet resulted in rapid precipitation of the free base. The fraction of the dose precipitating in the form of free base during dissolution is dependent on experimental factors such as stirring speed, which determines mixing kinetics of the highly supersaturated microenvironment solution into the bulk of the dissolution medium.

Thus, an oral solid dosage form of the Factor Xa inhibitor which does not have diminished oral bioavailability even when administered with antacids, H2 antagonists and proton pump inhibitors, which raise gastric pH to a pH at which the hydrochloride salt converts to the free base, would be a significant advance over traditional tablet formulations containing this drug.

### DESCRIPTION OF THE INVENTION

In accordance with the present invention, an oral solid dosage pharmaceutical composition is provided which includes a medicament which has the structure 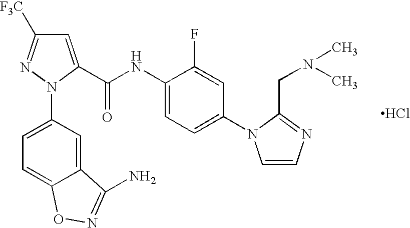
(hereinafter “the medicament” or “Factor Xa inhibitor”), and an acid to enhance dissolution of the medicament in the gastrointestinal tract under conditions of increased gastric pH.

Surprisingly, it has been found that the acid enhances oral bioavailability of the medicament in the presence of antacids, H2 antagonists and proton pump inhibitors. As indicated, presence of antacids, H2 antagonists and/or proton pump inhibitors with the medicament could increase gastric pH to greater than 5 which could result in conversion of the medicament in the form of a hydrochloride salt to the free base. The free base has very low aqueous solubility and dissolution rate and consequently low bioavailability.

In effect, the acid increases solubility of the Factor Xa inhibitor in the local environment of the dissolving solid dosage composition in the gastrointestinal tract resulting in an otherwise lower degree of supersaturation of the Factor Xa inhibitor local environment, than if the acid were not present. The result is that precipitation of the Factor Xa inhibitor in the form of its free base is minimized during dissolution of the Factor Xa inhibitor thereby increasing its oral bioavailability. The acid does not affect the pH of the bulk solution in the gastrointestinal tract and does not increase the solubility of the Factor Xa in this bulk solution. Rather, the acid improves dissolution behavior of Factor Xa mainly by preventing the precipitation of the insoluble free base during initial dissolution of the of Factor Xa.

Furthermore, in accordance with the present invention, a method for enhancing bioavailability of the Factor Xa inhibitor in the presence of antacids, H2 antagonists and proton pump inhibitors is also provided wherein an acid, such as tartaric acid, is incorporated with the solid dosage pharmaceutical carrier for the Factor Xa inhibitor. The acid will enhance dissolution of the Factor Xa inhibitor in the gastrointestinal tract even when gastric medium is at pH greater than 2.

The oral solid dosage pharmaceutical composition of the invention will include the medicament, acid, and conventional pharmaceutical excipients to enable formation of a pharmaceutically acceptable solid oral dosage form. The acid will be present in a molar ratio to the Factor Xa inhibitor compound within the range from about 0.1:1 to about 20:1, preferably from about 0.5:1 to about 10:1.

The composition of the invention will contain Factor Xa inhibitor compound in an amount within the range from about 1 to about 70% by weight and preferably from about 5 to about 50% by weight of the composition, and will contain the acid in an amount within the range from about 1 to about 50%, preferably from about 5 to about 30% by weight of the composition.

The composition of the invention will preferably contain
a) Factor Xa inhibitor compound (medicament);
b) at least one bulking agent;
c) preferably but optionally at least one binder;
d) preferably but optionally at least one disintegrant;
e) preferably but optionally at least one lubricant; and
f) at least one acid
   wherein
a) the medicament is present in an amount within the range from about 1 to about 70% by weight, preferably from about 5 to about 50% by weight;
b) the bulking agent is present in an amount within the range from about 2 to about 95% by weight, preferably from about 10 to about 85% by weight;
c) the binder is present in an amount within the range from about 0 to about 20% by weight, preferably from about 1 to about 10% by weight;
d) the disintegrant is present in an amount within the range from about 0 to about 20% by weight, and preferably from about 0.25 to about 15% by weight;
e) the lubricant is present in an amount within the range from about 0.1 to about 4% by weight, preferably from about 0.2 to about 2% by weight; and
f) the acid is present in an amount within the range from about 1 to about 50% by weight, preferably from about 5 to about 30% by weight; all of the above percentages being based on the total weight of the tablet.

It is preferrred that the bulking agent is microcrystalline cellulose;
the binder is hydroxypropyl cellulose;
the disintegrant is croscarmellose sodium;
the lubricant is magnesium stearate; and
the acid is tartaric acid, citric acid, succinic acid, malic acid, glycolic acid or adipic acid, preferably tartaric acid, or acidic salts thereof.

The formulations of this invention can be prepared by a variety of processes and order of addition of excipients. The utility of these formulations is not limited to a specific dosage form or manufacturing process. Capsules and tablets manufactured by wet granulation, dry granulation, direct blending or any other pharmaceutically acceptable process would show the same beneficial effect of the acids.

In accordance with the present invention, a preferred method is provided for preparing the composition of the invention which includes the steps of blending the Factor Xa inhibitor compound with one or more excipients such as bulking agent, binder and disintegrant, granulating the blend with water to form a wet granulation, drying the wet granulation, mixing the resulting dried granulation with acid to form acid-containing granulation, and forming the resulting acid-containing granulation into tablets or other solid dosage form. A lubricant will be preferably added to the acid-containing granulation to facilitate tablet formation. Tartaric acid is added to the granulation after drying and milling as an extragranular component. The incorporation of the acid in this manner is preferred to maximize dosage form stability and minimize chemical degradation.

### DETAILED DESCRIPTION OF THE INVENTION

The formulations of the invention may be in the form of immediate release solid dosage forms such as tablets, beads, beadlets, capsules, pills or sachets and may include granules and multi-layered tablets. In addition to the Factor Xa inhibitor compound and acid, the solid dosage forms of the invention may include one or more bulking agents or fillers, optionally one or more binders, optionally one or more disintegrants, optionally one or more lubricants, optionally one or more antiadherents and/or glidants, optionally one or more coatings.

In addition, in accordance with the present invention, a method for forming a solid pharmaceutical composition containing a Factor Xa inhibitor compound as described above having enhanced oral bioavailability even in the presence of antacids, H2 antagonists, and proton pump inhibitors, is provided, which includes the step of incorporating in the pharmaceutical composition an acid which increases solubility and prevents the conversion of the Factor Xa inhibitor in the gastrointestinal tract. The acid is as described above and is preferably tartaric acid.

The bulking agents or fillers will be present in the pharmaceutical compositions of the invention in an amount within the range from about 2 to about 95% by weight and preferably from about 10 to about 85% by weight of the composition. Examples of bulking agents or fillers suitable for use herein include, but are not limited to, cellulose derivatives such as microcrystalline cellulose or wood cellulose, lactose, sucrose, starch, pregelatinized starch, dextrose, mannitol, fructose, xylitol, sorbitol, corn starch, modified corn starch, inorganic salts such as calcium carbonate, calcium phosphate, dicalcium phosphate, calcium sulfate, dextrin/dextrates, maltidextrin, compressible sugars, and other known bulking agents or fillers, and/or mixtures of two or more thereof, preferably microcrystalline cellulose.

The binder will be optionally present in the pharmaceutical compositions of the invention in an amount within the range from about 0 to about 20% weight, preferably from about 1 to about 10% by weight of the composition. Examples of binders suitable for use herein include, but are not limited to, hydroxypropyl cellulose, corn starch, pregelatinized starch, modified corn starch, polyvinyl pyrrolidone (PVP) (molecular weight ranging from about 5,000 to about 80,000, preferably about 40,000), hydroxypropylmethyl cellulose (HPMC), lactose, gum acacia, ethyl cellulose, cellulose acetate, as well as a wax binder such as carnauba wax, paraffin, spermaceti, polyethylenes or microcrystalline wax, as well as other conventional binding agent and/or mixtures by two or more thereof, preferably hydroxypropyl cellulose.

The disintegrant will be optionally present in the pharmaceutical composition of the invention in an amount within the range from about 0 to about 20% by weight, preferably from about 0.25 to about 15% by weight of the composition. Examples of disintegrants suitable for use herein include, but are not limited to, croscarmellose sodium, crospovidone, potato starch, pregelatinized starch, corn starch, sodium starch glycolate, microcrystalline cellulose, or other known disintegrant, preferably croscarmellose sodium.

The lubricant will be optimally present in the pharmaceutical composition of the invention in an amount within the range from about 0.1 to about 4% by weight, preferably from about 0.2 to about 2% by weight of the composition. Examples of tableting lubricants suitable for use herein include, but are not limited to, magnesium stearate, zinc stearate, calcium stearate, talc, carnauba wax, stearic acid, palmitic acid, sodium stearyl fumarate or hydrogenated vegetable oils and fats, or other known tableting lubricants, and/or mixtures of two or more thereof, preferably magnesium stearate.

The acid employed in the pharmaceutical composition of the invention to enhance oral bioavailability in the gastrointestinal tract, even in the presence of antacids, H2 antagonists, and proton pump inhibitors, is preferably an organic carboxylic acid, although other organic or inorganic acids may be employed as well, and will be present in the pharmaceutical composition of the invention in an amount within the range from about 1 to about 50% by weight, preferably from about 5 to about 30% by weight of the composition. Examples of acids suitable for use herein include, but are not limited to, organic acids such as tartaric acid, citric acid, succinic acid, malic acid, glycolic acid, adipic acid, lactic acid, maleic acid, mandelic acid, propionic, glycine, glycerophosporic acid, any acid known to those skilled in the art and can be found, for example, in Remington's Pharmaceutical Sciences, 20th Edition, 2000, published by Mack publishing Company, Eaton, Pa., and/or mixtures of two or more of those acids, preferably tartaric acid.

Preferred formulations of the invention are set out below.

| Ingredient | Preferred Range | More Preferred Range |
|---|---|---|
| Factor XA compound | 5-50% | 6-25% |
| Fillers or Bulking Agents | 10-85% | 40-80% |
| Disintegrants | 0.25-15% | 0.5-5% |
| Lubricants | 0.2-2% | 0.3-0.75% |
| Acid | 5-30% | 10-22% |

More preferred formulations of the invention are set out below.

| Ingredient | Preferred Range | More Preferred Range |
|---|---|---|
| Factor Xa compound | 5-50% | 6-25% |
| Microcrystalline Cellulose | 10-85% | 40-80% |
| Hydroxypropyl Cellulose | 1-10% | 2-5% |
| Croscarmellose Sodium | 0.25-15% | 0.5-5% |
| Magnesium Stearate | 0.2-2% | 0.3-0.75% |
| Acid (preferably tartaric acid) | 5-30% | 10-22% |

Other conventional ingredients which may optionally be present in compositions of the invention include stabilizers, anti-adherents or silica flow conditioners or glidants, such as Cab-O-Sil or Syloid brand silicon dioxide as well as antioxidants such as Vitamin E, Vitamin C, and folic acid, Vitamin B₆ and Vitamin B₁₂.

The tablet of the invention may also include an outer protective coating layer which may comprise from 0 to about 15% by weight of the tablet. The outer protective coating layer which is applied over the tablet may comprise any conventional coating formulations and will include one or more film-formers or binders, such as a hydrophilic polymer like hydroxypropylmethyl cellulose (HPMC), a disaccharide like lactose, a hydrophobic polymer like ethyl cellulose, cellulose acetate, polyvinyl alcohol-maleic anhydride copolymers, acrylic copolymers, β-pinene polymers, glyceryl esters of wood resins and the like, and one or more plasticizers, such as polyethylene glycol, triethyl citrate, diethyl phthalate, propylene glycol, glycerin, butyl phthalate, castor oil and the like.

The film formers are applied from a solvent system containing one or more solvents including water, alcohols like ethyl alcohol or isopropyl alcohol, ketones like acetone, or ethylmethyl ketone, chlorinated hydrocarbons like methylene chloride, dichloroethane, and 1,1,1-trichloroethane.

In carrying out the method of the present invention, the pharmaceutical composition of the invention may be administered to mammalian species, such as monkeys, dogs, cats, rats, humans, etc., and, as described hereinbefore, may be incorporated in a tablet or capsule or other solid dosage form. The above dosage forms also include the necessary carrier materials, excipients, lubricant, antibacterial, and optionally anti-oxidants such as Vitamin C and Vitamin E, as well as Vitamin B₆, Vitamin B₁₂, folic acid, sodium bisulfite, and the like.

The dose administered must be adjusted according to age, weight and condition of the patient, as well as the route of administration, dosage form and regimen and the desired result.

The compositions described above may be administered in the dosage forms as described above in single or divided doses of one to four times daily. It may be advisable to start a patient on a low dose combination and work up gradually to a high dose combination.

Tablets of various sizes can be prepared, e.g., of about 20 to 2000 mg in total weight, containing the active substances in the ranges described above, with the remainder being a physiologically acceptable carrier of other materials according to accepted pharmaceutical practice. These tablets can, of course, be scored to provide for fractional doses in some cases. Gelatin capsules can be similarly formulated.

The formulations as described above will be administered for a prolonged period, that is, for as long as the potential for diseases involving Factor Xa. Sustained release forms of such formulations which may provide such amounts daily, biweekly, weekly, and monthly and the like may also be employed. A dosing period of at least 10 days are required to achieve minimal benefit.

### BRIEF DESCRIPTION OF THE FIGURES

FIG. 1 is a graph showing in vitro dissolution profiles in acetate buffer, pH 5.5, of Factor Xa inhibitor tablets (Examples 1A, 2A and 3A) which do not contain acids;

FIG. 2 is a graph showing in vitro dissolution profiles in acetate buffer, pH 5.5, of Factor Xa inhibitor tablets each containing an acid (Example 1—tartaric acid, Example 2—tartaric acid and Example 3—citric acid) in accordance with the present invention;

FIG. 3 is a graph showing in vitro dissolution profiles in acetate buffer, pH 5.5, of Factor Xa inhibitor tablets each containing an acid (Example 4—succinic acid, Example 5—malic acid and Example 6—glycolic acid) in accordance with the present invention;

FIG. 4 is a graph showing in vitro dissolution profiles in acetate buffer, pH 5.5, of Factor Xa inhibitor tablets each containing an acid (Example 7—adipic acid and Example 8—tartaric acid) in accordance with the present invention;

FIG. 5 is a graph showing plasma profiles in dogs obtained after administration of tablets from Example 3A (no acid); and Example 1 (tartaric acid); and

FIG. 6 is a graph showing in vitro dissolution in acetate buffer, pH 5.5, of Factor Xa inhibitor tablets each containing an acid (Example 9—tartaric acid) in accordance with the present invention.

### EXAMPLES

The following Examples represent preferred embodiments of the present invention.

### Example 1

Immediate release tablets having the following composition in accordance with the present invention were prepared as described below.

Formula

| Tablet | Dry Milled | | |
|---|---|---|---|
| (% by | Granulation | | |
| weight) | (% by wt.) | Ingredient | |
| 82.9% | 35.81 | Factor Xa inhibitor | Dry Milled |
| (milled | | (hydrochloride salt) | Granulation |
| granulation) | 60.67 | Microcrystalline | |
| | | Cellulose, NF | |
| | 3.02 | Hydroxypropyl Cellulose, NF | |
| | | (Klucel ® LF) | |
| | 0.5 | Croscarmellose Sodium, NF | |
| 0.5% | | Magnesium Stearate, NF | |
| 16.6% (acid) | | Tartaric acid, NF | |

The Factor Xa inhibitor compound was blended with microcrystalline cellulose, croscarmellose sodium and hydroxypropyl cellulose (Klucel LF) in a high shear granulation/mixer. The blend was then granulated with water in the high shear mixer. The resulting wet granulation was screened through a 6-mesh screen and dried in a hot air convection oven at 50° C. to a moisture content of 3%. The dried granulation was milled using a 20-mesh screen, blended with tartaric acid, and then lubricated by blending with magnesium stearate using a Turbula® mixer. The lubricated blend was compressed using a single station press into 100 mg (free base equivalent) tablets of the invention.

### Example 1A

### (Control—no Acid)

Immediate release tablets having the following composition were prepared as described below.

Formula

| Amount | |
|---|---|
| (% by wt.) | Ingredient |
| 35.63 | Factor Xa inhibitor (hydrochloride salt) |
| 60.37 | Microcrystalline Cellulose, NF |
| 3.0 | Hydroxypropyl Cellulose, NF (Klucel ® LF) |
| 0.5 | Croscarmellose Sodium, NF |
| 0.5 | Magnesium Stearate, NF |

The Factor Xa inhibitor was blended with microcrystalline cellulose, croscarmellose sodium and hydroxypropyl cellulose (Klucel LF) in a high shear granulator/mixer. The blend was then granulated with water in the high shear mixer. The resulting wet granulation was screened through a 6-mesh screen and dried in a hot air convection oven at 50° C. to a moisture content of 3%. The dried granulation was milled using a 20-mesh screen and then lubricated by blending with magnesium stearate. The lubricated blend was compressed into 100 mg (free base equivalent) tablets using a Carver® press.

### Example 2

Immediate release tablets having the following composition in accordance with the present invention were prepared as described below.

Formula

| Tablet | Lubricated | |
|---|---|---|
| (% by | Granulation | |
| weight) | (% by wt.) | Ingredient |
| 83.3% | 35.63 | Factor Xa inhibitor (hydrochloride salt) |
| (lubricated | 60.37 | Microcrystalline Cellulose, NF |
| blend) | 3.0 | Hydroxypropyl Cellulose, NF (Klucel ® LF) |
| | 0.5 | Croscarmellose Sodium, NF |
| | 0.5 | Magnesium Stearate, NF |
| 16.7% (acid) | | Tartaric acid, NF |

The Factor Xa inhibitor compound was blended with microcrystalline cellulose, croscarmellose sodium and hydroxypropyl cellulose (Klucel LF) in a high shear granulator/mixer. The blend was then granulated with water in the high shear mixer. The resulting wet granulation was screened through a 6-mesh screen and dried in a hot air convection oven at 50° C. to a moisture content of 3%. The dried granulation was milled using a 20-mesh screen and then lubricated by blending with magnesium stearate. The lubricated blend was blended with tartaric acid. Tablets, 100 mg (free base equivalent), were formed by compressing the lubricated blend containing tartaric acid, using a Carver® press.

### Example 2A

### (Control—no Acid)

Immediate release film coated tablets having the following composition were prepared as described below.

Formula

| Amount | |
|---|---|
| (% by wt.) | Ingredient |
| Core Tablet | |
| 35.63 | Factor Xa compound (hydrochloride salt) |
| 60.37 | Microcrystalline Cellulose, NF |
| 3.0 | Hydroxypropyl Cellulose, NF (Klucel ® LF) |
| 0.5 | Croscarmellose Sodium, NF |
| 0.5 | Magnesium Stearate, NF |
| Film Coat | |
| 4.0^{(a)} | Opadry ® II White (HPMC-based coating |
| | formulation) |
| 0.5^{(a)} | Opadry ® Clear (HPMC-based coating |
| | formulation) |
| 0.02^{(a)} | Carnauba Wax, NF |

^{(a)}Represents weight gain based on the core tablet weight.

The Factor Xa inhibitor compound was blended with microcrystalline cellulose, croscarmellose sodium and ⅓ the quantity of hydroxypropyl cellulose (Klucel® LF) in a high shear granulator/mixer. The remaining amount of hydroxypropyl cellulose (Klucel® LF) was dissolved in water to form a 6% (w/w) solution and used to granulate the blend in the high shear mixer. Additional water was used to complete the granulation process and achieve the desired end point. The resulting wet granulation was dried in a hot air convection oven at 50° C. to a moisture content of NMT 3%. The dried granulation was milled using a 20-mesh screen and then lubricated by blending with magnesium stearate. The lubricated blend was compressed into 25 mg (free base equivalent) tablets using a rotary tablet press. Opadry® II White was applied to the tablets in a coating pan, followed by Opadry® Clear and carnauba wax to form the film coated tablets.

### Example 3

Immediate release tablets having the following composition in accordance with the present invention were prepared as described below.

| Tablet | Lubricated | |
|---|---|---|
| (% by | Granulation | |
| weight) | (% by wt.) | Ingredient |
| 83.3% | 35.63 | Factor Xa inhibitor (hydrochloride salt) |
| (lubricated | 60.37 | Microcrystalline Cellulose, NF |
| blend) | 3.0 | Hydroxypropyl Cellulose, NF (Klucel ® LF) |
| | 0.5 | Croscarmellose Sodium, NF |
| | 0.5 | Magnesium Stearate, NF |
| 16.7% (acid) | | Citric acid, anhydrous |

The Factor Xa inhibitor compound was blended with microcrystalline cellulose, croscarmellose sodium and hydroxypropyl cellulose (Klucel LF) in a high shear granulator/mixer. The blend was then granulated with water in the high shear mixer. The resulting wet granulation was screened through a 6-mesh screen and dried in a hot air convection oven at 50° C. to a moisture content of 3%. The dried granulation was milled using a 20-mesh screen and then lubricated by blending with magnesium stearate. The lubricated blend was blended with citric acid, anhydrous. Tablets, 100 mg (free base equivalent), were formed by compressing the lubricated blend containing citric acid, using a single station press.

### Example 3A

### (Control—no Acid)

Immediate release film coated tablets having the following composition were prepared as described below.

Formula

| Amount | |
|---|---|
| (% by wt.) | Ingredient |
| Core Tablet | |
| 35.63 | Factor Xa compound (hydrochloride salt) |
| 60.37 | Microcrystalline Cellulose, NF |
| 3.0 | Hydroxypropyl Cellulose, NF (Klucel ® LF) |
| 0.5 | Croscarmellose Sodium, NF |
| 0.5 | Magnesium Stearate, NF |
| Film Coat | |
| 3.5^{(a)} | Opadry ® II White (HPMC-based coating |
| | formulation) |
| 0.5^{(a)} | Opadry ® Clear (HPMC-based coating |
| | formulation) |
| 0.02^{(a)} | Carnauba Wax, NF |

^{(a)}Represents weight gain based on the core tablet weight.

The Factor Xa inhibitor compound was blended with microcrystalline cellulose, croscarmellose sodium and ⅓ the quantity of hydroxypropyl cellulose (Klucel® LF) in a high shear granulator/mixer. The remaining amount of hydroxypropyl cellulose (Klucel® LF) was dissolved in water to form a 6% (w/w) solution and used to granulate the blend in the high shear mixer. Additional water was used to complete the granulation process and achieve the desired end point. The wet granulation was dried in a hot air convection oven at 50° C. to a moisture content of NMT 3%. The dried granulation was milled using a 20-mesh screen and then lubricated by blending with magnesium stearate. The lubricated blend was compressed into 50 mg (free base equivalent) tablets using a rotary tablet press. Opadry® II White was applied to the tablets in a coating pan, followed by Opadry® Clear and carnauba wax to form the film coated tablets.

### Example 4

Immediate release tablets having the following composition in accordance with the present invention were prepared as described below.

| Tablet | Lubricated | |
|---|---|---|
| (% by | Granulation | |
| weight) | (% by wt.) | Ingredient |
| 83.3% | 35.63 | Factor Xa inhibitor (hydrochloride salt) |
| (lubricated | 60.37 | Microcrystalline Cellulose, NF |
| blend) | 3.0 | Hydroxypropyl Cellulose, NF (Klucel ® LF) |
| | 0.5 | Croscarmellose Sodium, NF |
| | 0.5 | Magnesium Stearate, NF |
| 16.7% (acid) | | Succinic acid |

The Factor Xa inhibitor compound was blended with microcrystalline cellulose, croscarmellose sodium and hydroxypropyl cellulose (Klucel LF) in a high shear granulator/mixer. The blend was then granulated with water in the high shear mixer. The resulting wet granulation was screened through a 6-mesh screen and dried in a hot air convection oven at 50° C. to a moisture content of 3%. The dried granulation was milled using a 20-mesh screen and then lubricated by blending with magnesium stearate. The lubricated blend was blended with succinic acid using a Turbula® mixer. Tablets, 100 mg (free base equivalent), were formed by compressing the lubricated blend containing succinic acid, using a Carver® press.

### Example 5

Immediate release tablets having the following composition in accordance with the present invention were prepared as described below.

| Tablet | Lubricated | |
|---|---|---|
| (% by | Granulation | |
| weight) | (% by wt.) | Ingredient |
| 83.3% | 35.63 | Factor Xa inhibitor (hydrochloride salt) |
| (lubricated | 60.37 | Microcrystalline Cellulose, NF |
| blend) | 3.0 | Hydroxypropyl Cellulose, NF (Klucel ® LF) |
| | 0.5 | Croscarmellose Sodium, NF |
| | 0.5 | Magnesium Stearate, NF |
| 16.7% (acid) | | Malic acid |

The Factor Xa inhibitor compound was blended with microcrystalline cellulose, croscarmellose sodium and hydroxypropyl cellulose (Klucel LF) in a high shear granulator/mixer. The blend was then granulated with water in the high shear mixer. The resulting wet granulation was screened through a 6-mesh screen and dried in a hot air convection oven at 50° C. to a moisture content of 3%. The dried granulation was milled using a 20-mesh screen and then lubricated by blending with magnesium stearate. The lubricated blend was blended with malic acid. Tablets, 100 mg (free base equivalent), were formed by compressing the lubricated blend containing malic acid, using a Carver® press.

### Example 6

Immediate release tablets having the following composition in accordance with the present invention were prepared as described below.

| Tablet | Lubricated | |
|---|---|---|
| (% | Granulation | |
| by weight) | (% by wt.) | Ingredient |
| 83.3% | 35.63 | Factor Xa inhibitor (hydrochloride salt) |
| (lubricated | 60.37 | Microcrystalline Cellulose, NF |
| blend) | 3.0 | Hydroxypropyl Cellulose, NF (Klucel ® LF) |
| | 0.5 | Croscarmellose Sodium, NF |
| | 0.5 | Magnesium Stearate, NF |
| 16.7% | | Glycolic acid |

The Factor Xa inhibitor compound was blended with microcrystalline cellulose, croscarmellose sodium and hydroxypropyl cellulose (Klucel LF) in a high shear granulator/mixer. The blend was then granulated with water in the high shear mixer. The resulting wet granulation was screened through a 6-mesh screen and dried in a hot air convection oven at 50° C. to a moisture content of 3%. The dried granulation was milled using a 20-mesh screen and then lubricated by blending with magnesium stearate. The lubricated blend was blended with glycolic acid using a Turbula® mixer. Tablets, 100 mg (free base equivalent), were formed by compressing the lubricated blend containing glycolic acid, using a Carver® press.

### Example 7

Immediate release tablets having the following composition in accordance with the present invention were prepared as described below.

| | Lubricated | |
|---|---|---|
| Tablet (% | Granulation | |
| by weight) | (% by wt.) | Ingredient |
| 83.3% | 35.63 | Factor Xa inhibitor (hydrochloride salt) |
| (lubricated | 60.37 | Microcrystalline Cellulose, NF |
| blend) | 3.0 | Hydroxypropyl Cellulose, NF (Klucel ® LF) |
| | 0.5 | Croscarmellose Sodium, NF |
| | 0.5 | Magnesium Stearate, NF |
| 16.7% | | Adipic acid |

The Factor Xa inhibitor compound was blended with microcrystalline cellulose, croscarmellose sodium and hydroxypropyl cellulose (Klucel LF) in a high shear granulator/mixer. The blend was then granulated with water in the high shear mixer. The resulting wet granulation was screened through a 6-mesh screen and dried in a hot air convection oven at 50° C. to a moisture content of 3%. The dried granulation was milled using a 20-mesh screen and then lubricated by blending with magnesium stearate. The lubricated blend was blended with adipic acid. Tablets, 100 mg (free base equivalent), were formed by compressing the lubricated blend containing adipic acid, using a Carver® press.

### Example 8

Immediate release tablets having the following composition were prepared as described below where tartaric acid is added before adding Mg stearate lubricant.

Formula

| Amount (% by wt.) | Ingredient |
|---|---|
| 29.70 | Factor Xa inhibitor (hydrochloride salt) |
| 49.63 | Microcrystalline Cellulose, NF |
| 3.0 | Hydroxypropyl Cellulose, |
| | NF (Klucel ® LF) |
| 0.5 | Croscarmellose Sodium, NF |
| 16.67 | Tartaric acid, NF |
| 0.5 | Magnesium Stearate, NF |

The Factor Xa inhibitor was blended with microcrystalline cellulose, croscarmellose sodium and hydroxypropyl cellulose (Klucel LF) in a high shear granulator/mixer. The blend was then granulated with water in the high shear mixer. The resulting wet granulation was dried in a hot air convection oven at 50° C. to a moisture content of NMT 3%. The dried granulation was milled using a 20-mesh screen and blended with tartaric acid and then lubricated by blending with magnesium stearate. The lubricated blend was compressed into 25 mg (free base equivalent) tablets using a rotary tablet press.

### Example 9

Immediate release tablets having the following composition were prepared as described below.

Formula

| Amount | |
|---|---|
| (% by wt.) | Ingredient |
| 14.85 | Factor Xa compound (hydrochloride salt) |
| 62.98 | Microcrystalline Cellulose, NF |
| 3.0 | Hydroxypropyl Cellulose, NF (Klucel ® LF) |
| 1.5 | Croscarmellose Sodium, NF |
| 16.67 | Tartaric acid, NF |
| 0.5 | Colloidal Silicon Dioxide, NF |
| 0.5 | Magnesium Stearate, NF |

The Factor Xa inhibitor compound was blended with microcrystalline cellulose, hydroxypropyl cellulose (Klucel® LF) and ⅓ the quantity of hydroxypropyl croscarmellose sodium in a high shear granulator/mixer. The blend was then granulated with water in the high shear mixer. The resulting wet granulation was dried in a hot air convection oven at 50° C. to a moisture content of NMT 3.5%. The dried granulation was milled using a conical screen mill and a 20-mesh screen. The milled granulation was blended with tartaric acid, colloidal silicon dioxide and the remaining amount of croscarmellose sodium and then lubricated by blending with magnesium stearate. The lubricated blend was compressed into 12.5 mg (free base equivalent) tablets using a rotary tablet press.

### Example 10

### Dissolution Studies

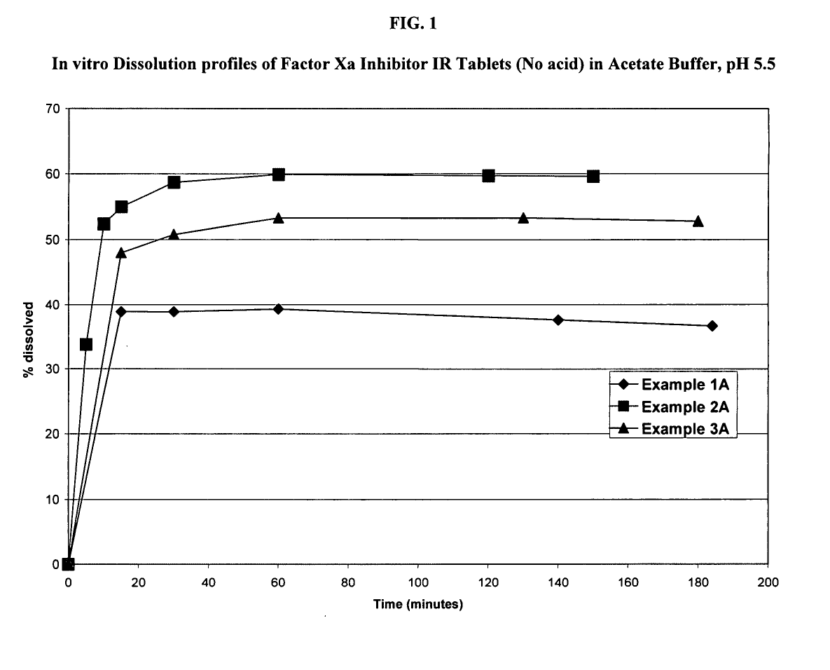

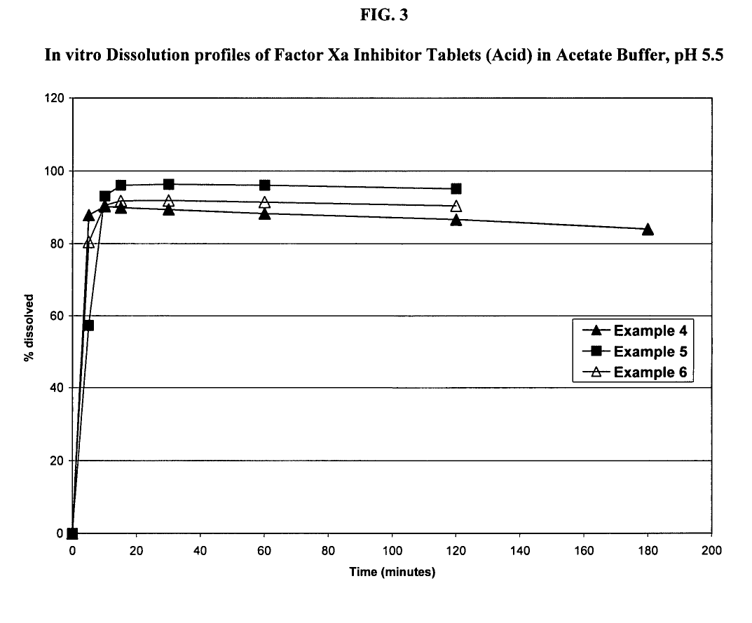

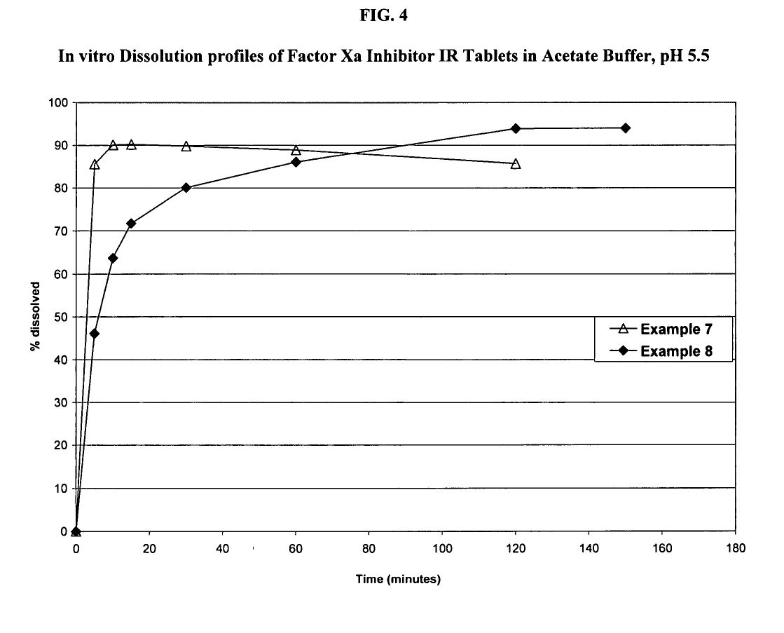

In vitro dissolution studies were carried out to show that addition of an acid to a tablet formulation (in accordance with the present invention) as described in Examples 1 to 8 increased fraction of the dosed dissolved at the end of the test over tablet formulations which did not contain added acid (Examples 1A, 2A and 3A). The dissolution studies were conducted using USP apparatus 2 in acetate buffer, pH 5.5 and 37° C. The results obtained are shown in FIGS. 1 to 4.

A brief description of each of the tablet formulations tested is set out below.

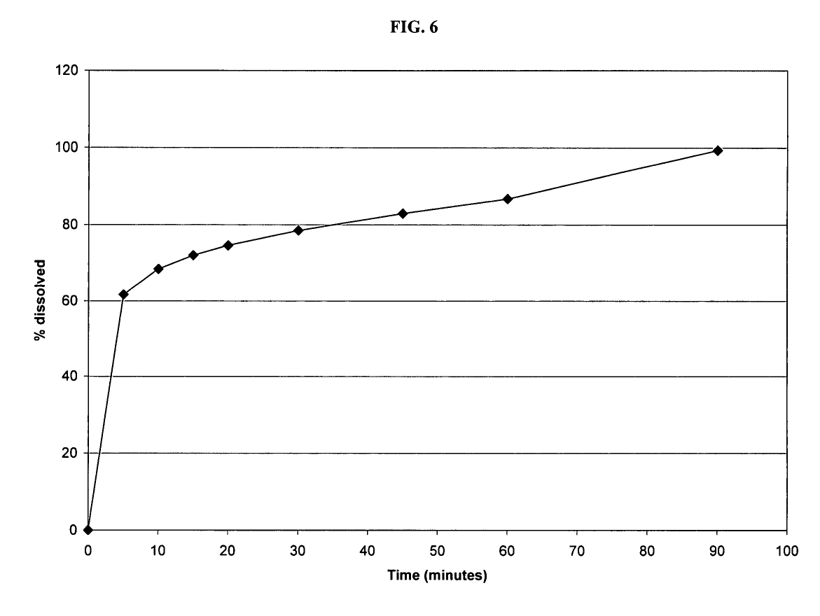

Tablet formations tested for in vitro dissolution properties:
Example 1A tablets similar to Example 1 tablets but with no added acid;
Examples 2A and 3A tablets similar to Examples 1, 2 and 3 but containing a film coat and no added acid;
Example 1 tablets—contains tartaric acid blended with dry milled granulation;
Example 2 tablets—contains tartaric acid blended with lubricated granulation;
Example 3 tablets—contains citric acid blended with lubricated granulation;
Example 4 tablets—contains succinic acid blended with lubricated granulation;
Example 5 tablets—contains malic acid blended with lubricated granulation;
Example 6 tablets—contains glycolic acid blended with lubricated granulation;
Example 7 tablets—contains adipic acid blended with lubricated granulation;
Example 8 tablets—contains tartaric acid blended with dry milled granulation;
The use of acidic components, such as tartaric acid, in the tablet formulation of the invention resulted in significant improvement in the in-vitro performance of the tablets at increased pH condition. In-vitro dissolution profile of the tablets containing tartaric acid showed a rapid dissolution rate in the pH 5.5 buffer and the fraction dissolved ranged between 85-100%, in contrast to 35-40% for the tablets without tartaric acid under the same experimental conditions (FIGS. 1, 2, 4 and 6). The pH of the dissolution medium remained substantially constant at ˜5.5 until the end of the test. Thus, tartaric acid does not enhance the dissolution behavior of the Factor Xa inhibitor by increasing its solubility in the bulk medium. Instead, it increases the solubility in the local environment of the dissolving dosage form, resulting in a lower degree of supersaturation in such environment and thus minimizing free base precipitation during dissolution of the hydrochloride salt. Once diluted into the bulk medium, the drug is capable of remaining in supersaturated solution for a sufficient time period to allow for drug absorption to take place.

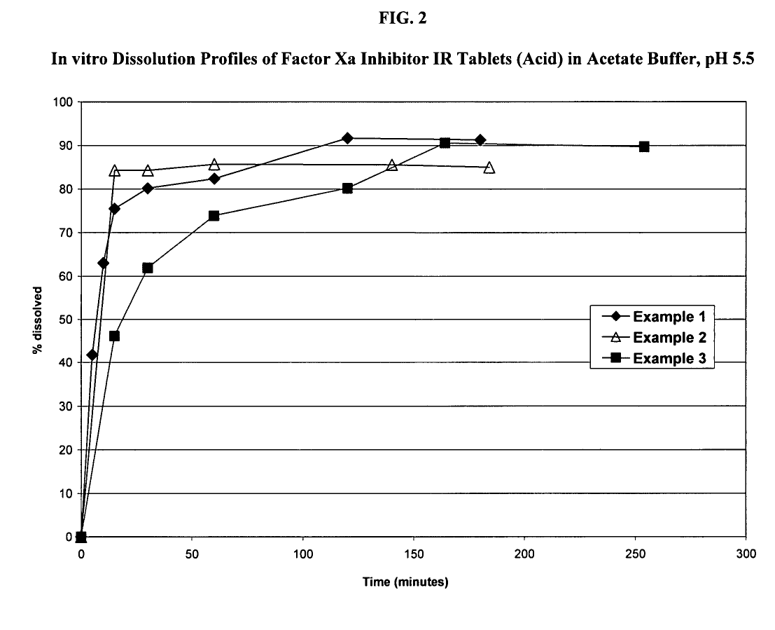

The use of other organic or inorganic acids, such as citric and succinic acids (FIGS. 2 to 4), also resulted in enhancement of the Factor Xa inhibitor in-vitro dissolution profile at pH 5.5.

### Dog Studies

Formulations (Examples 1, 3A) of the Factor Xa inhibitor were tested in vivo in a canine model for its pharmacokinetic profiles. In order to evaluate formulations in dogs, the emphasis was focused on the absorption phase and in vitro/in vivo correlation (IVIVC). Many factors affect a drug's in vivo dissolution, solubility, and permeability in the stomach and intestine. These factors include gastric fluid volume, gavage volume, gastric pH, in vivo dissolution, pH-dependent drug solubility, and food effects.

Therefore, the following three factors were controlled in this animal model: First, canine gastric pH was maintained by either pentagastrin treatment or famotidine treatment. Since canine gastric pH is higher and more variable than human, the pH variability in dogs affects the solubility and dissolution rate of certain drugs with pH-dependent solubility profiles, especially for weak base compounds with a pKa 3-8 and low solubility under high pH. Thus, pentagastrin (6 μg/kg) was given intramuscularly 30 minutes before dosing of the Factor Xa inhibitor formulations (Examples 1, 3A); This maintains the canine gastric pH at 2-3 for 1 hour to allow adequate time for complete dissolution. On other hand, famotidine (40 mg/dog) was given orally 3 hours before dosing; and famotidine maintains the canine gastric pH at 5-7 for more than several hours to allow enough dosing time window. Second, gavage volume of water was standardized in all animal studies. A total of 50 ml water was given by gavage after the formulation administration in the studies. In addition, animals were denied access of water 1 hour prior to and post dosing to control the gastric fluid volume. This standard volume was used to give adequate volume for complete dissolution of formulations in vivo and to achieve uniform IVIVC. Third, in order to eliminate the food effect on lipid content, drug solubility, and dissolution in vivo, the animals were fasted overnight until 4 hours post dosing.

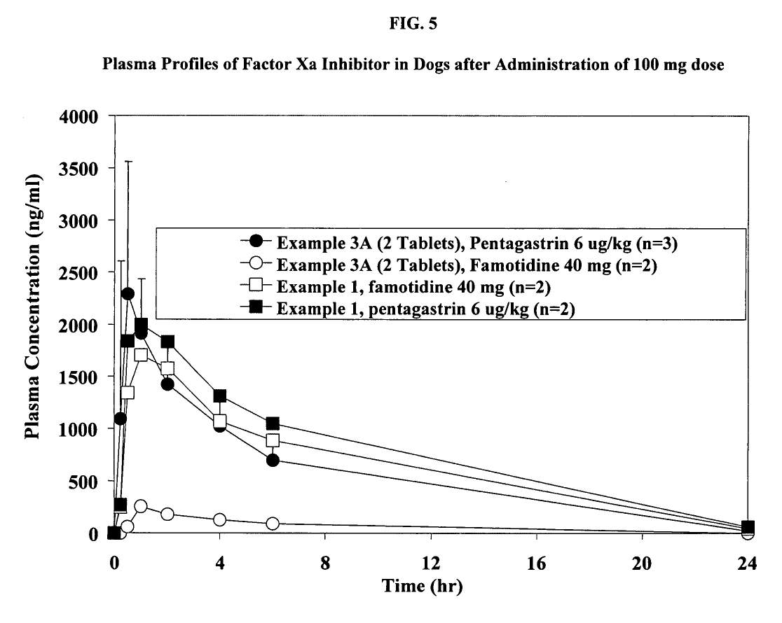

Plasma profiles of Factor Xa inhibitor in dogs after administration of tablets from Example 3A (no acid) and Example 1 (tartaric acid) are shown in FIG. 5. Compared to pentagastrin treatment, famotidine (40 mg) treatment decreased the Cmax of the factor Xa inhibitor formulation (Example 3A, 100 mg; formulation without acid) by 85% from 2413 ng/ml to 257 ng/ml, and decreased its AUC0-24 by 88% from 10716 ng/ml*h to 1143 ng/ml*h in dogs. However, famotidine (40 mg) treatment did not significantly decrease the Cmax and AUC0-24 of the factor Xa inhibitor formulation (Example 1, 100 mg; formulation containing tartaric acid) compared to pentagastrin treatment in dogs. Although the Cmax of Example 1 decreased by 19.5% from 2136 ng/ml to 1720 ng/ml, and the AUC0-24 of Example 1 decreased by 28.7% from 14408 ng/ml*h to 10270 ng/ml*h after famotidine treatment, these differences are not statistically significant from the values obtained in pentagastrin treated dogs. This indicates that formulation (Example 1) overcomes the pH-dependent absorption of the Factor Xa inhibitor in vivo.

1. An oral solid dosage pharmaceutical composition comprising a medicament which has the structure 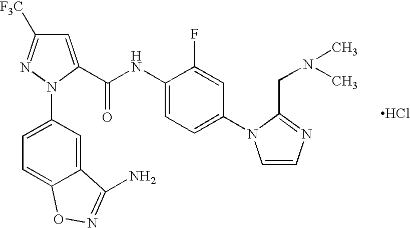
and an acid to enhance dissolution of the medicament in the gastrointestinal tract.

2. The composition as defined in claim 1 wherein the acid lowers the pH of the environment of the composition once gastrointestinal fluid enters the composition, thereby causing dissolution of the medicament, and the acid enhances dissolution and bioavailability of the medicament when co-administered with antacids H2 antagonists, and proton pump inhibitors.

3 The composition as defined in claim 1 wherein the acid is an organic carboxylic acid.

4 The composition as defined in claim 3 wherein the acid is tartaric acid, citric acid, succinic acid, malic acid, glycolic acid or adipic acid.

5. The composition as defined in claim 1 wherein the acid is an inorganic acid.

6. The composition as defined in claim 1 in the form of an immediate release tablet, capsule or beadlet.

7. The composition as defined in claim 1 wherein the acid is present in a molar ratio to the medicament within the range from about 0.1:1 to about 20:1.

8. The composition as defined in claim 1 containing from about 1 to about 70% by weight of the medicament, and the acid is present in an amount within the range from about 1 to about 50% by weight of the composition.

9. The composition as defined in claim 1 in the form of a tablet comprising:
a) medicament;
b) at least one bulking agent;
c) optionally at least one binder;
d) optionally at least one disintegrant;
e) optionally at least one lubricant; and
f) at least one acid.

10. The composition as defined in claim 9 wherein:
a) the medicament is present in an amount within the range from about 1 to about 70% by weight;
b) the bulking agent is present in an amount within the range from about 2 to about 95% by weight;
c) the binder is present in an amount within the range from about 0 to about 20% by weight;
d) the disintegrant is present in an amount within the range from about 0 to about 20% by weight;
e) the lubricant is present in an amount within the range from about 0.1 to about 4% by weight; and
f) the acid is present in an amount within the range from about 1 to about 50% by weight.

11. The composition as defined in claim 10 wherein
the bulking agent is microcrystalline cellulose;
the binder is hydroxypropyl cellulose;
the disintegrant is croscarmellose sodium;
the lubricant is magnesium stearate; and
the acid is tartaric acid, citric acid, succinic acid, malic acid, glycolic acid or adipic acid.

12. The composition as defined in claim 111 wherein the acid is tartaric acid.

13. The composition as defined in claim 1 in the form of an immediate release tablet comprising

| Amount (% by wt.) | |
|---|---|
| Tablet | Ingredient |
| 14.85 | Medicament |
| 62.98 | Microcrystalline Cellulose, NF |
| 3.0 | Hydroxypropyl Cellulose, |
| | NF (Klucel ® LF) |
| 1.5 | Croscarmellose Sodium, NF |
| 16.67 | Tartaric acid, NF |
| 0.5 | Colloidal Silicon Dioxide, NF |
| 0.5 | Magnesium Stearate, NF |

14. A method for forming a solid pharmaceutical composition comprising a Factor Xa inhibitor having the structure 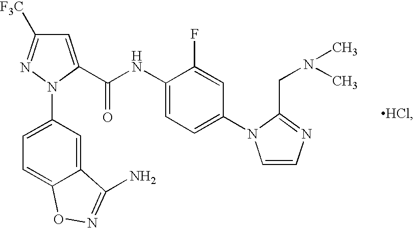
having enhanced oral bioavailability in the presence of antacids, H2 antagonists, and proton pump inhibitors, which comprises incorporating in the pharmaceutical composition an acid.

15. The method as defined in claim 14 wherein the acid is tartaric acid.

16. A method for preparing the pharmaceutical composition as defined in claim 1 in the form of a tablet, which comprises:
a) blending the Factor Xa inhibitor compound with one or more excipients and water to form a wet granulation;
b) drying the wet granulation;
c) mixing the resulting dried granulation with an acid; and
d) forming the resulting blend into tablets.

17. The method as defined in claim 16 wherein the pharmaceutical composition is an immediate release composition.

18. The method as defined in claim 17 wherein the acid is an organic carboxylic acid which is tartaric acid, citric acid, succinic acid, malic acid, glycolic acid or adipic acid.

19. The method as defined in claim 17 wherein the acid is tartaric acid.
